(19) United States
(12) Patent Application Publication
(10) Pub. No.: US 2021/0106455 A1
(43) Pub. Date: Apr. 15, 2021

(54) NASAL BREATHING TRAINING TAPE AND METHODFIELD OF THE INVENTION

(71) Applicant: Patrick McKeown, Co. Galway (IE)

(72) Inventor: Patrick McKeown, Co. Galway (IE)

(21) Appl. No.: 17/079,472

(22) Filed: Oct. 24, 2020

Related U.S. Application Data
(63) Continuation-in-part of application No. 16/542,363, filed on Aug. 16, 2019.

(51) Int. Cl. A61F 5/56

(52) U.S. Cl. CPC A61F 5/56

(57) ABSTRACT

Disclosed is a nasal breathing training tape adapted to impart training to a user to breathe from the nose, and not from the mouth, during sleep time or wakefulness time. The nasal breathing training tape includes a stretchable strip, an adhesive layer, and a cutout portion. The stretchable strip includes a first surface, and a second surface opposite to the first surface. The stretchable strip is stretchable to create tension therein. The adhesive layer may be applied to the second surface of the stretchable strip. The cutout portion is configured on the stretchable strip along a substantially central location of the stretchable strip. The stretchable strip is stretched to a size to be accommodated and stick around a upper and lower lips, surrounding the mouth but not touching the upper and lower lips, of the user, such that the upper and lower lips are held together by the tension created in the stretchable strip.

The present invention generally relates to training equipment, and, more particularly, to a training equipment that imparts training to children, teens and adults to breathe from the nose and not from the mouth, during sleep time or wakefulness time.

### BACKGROUND OF THE INVENTION

The inventor, Patrick McKeown has written seven books on breathing re-education for children, teenagers and adults. The foundation of breathing re-education is to stop the habit of breathing through the mouth during wakefulness and sleep and change to nasal breathing only. Human beings, particularly children and teenagers, have a common tendency to breathe from their mouth rather than their nose, during wakefulness while performing various day to day activities, such as playing, sitting, watching television or during sleep. Up to 50% of studied children persistently breathe through an open mouth. (Felcar, Josiane Marques, Izabele Rafael Bueno, Ana Carolina Silva Massan, Roberta Pereira Torezan, and Jefferson Rosa Cardoso. “Prevalence of mouth breathing in children from an elementary school.” Ciencia & saude coletiva 15, no. 2 (2010): 427.) Breathing through the mouth is not natural and can cause various problems to both children and adults. For example, mouth breathing can contribute to crooked teeth, facial deformities, drooling, or poor growth of children. In adults, chronic mouth breathing can cause bad breath, gum disease and can worsen symptoms of other illnesses including asthma and sleep apnea. (McKeown P, The Oxygen Advantage. Little Brown Press. September 2015).

Mouth breathing during sleep is significantly associated with worse oxygen desaturation and an increased degree of upper airway collapse in adults. (Yen-Bin Hsu, Association Between Breathing Route, Oxygen Desaturation, and Upper Airway Morphology. DOI: 10.1002/lary.28774)

Accordingly, there exists a need to impart training to children, teenagers or adults to breathe from the nose and not from the mouth, during sleep time and wakefulness.

### SUMMARY OF THE INVENTION

In view of the foregoing disadvantages inherent in the prior art, the general purpose of the present invention is to provide a nasal breathing training tape and method to overcome the drawbacks inherent in the prior art.

An object of the present disclosure is to impart training skills to children, teenagers or adults to breathe from the nose and not from the mouth, during sleep time or wakefulness.

With that regard, in one aspect of the present invention, a nasal breathing training tape is provided. The nasal breathing training tape is adapted to impart training to a user to breathe from the nose and not from the mouth, during sleep time or wakefulness. The nasal breathing training tape includes a stretchable strip, an adhesive layer, and a cutout portion. The stretchable strip includes a first surface, and a second surface opposed to the first surface. The stretchable strip has sufficient stretch to create tension therein. Further, the adhesive layer may be applied to the second surface of the stretchable strip. Furthermore, the cutout portion is disposed along a substantially central location of the stretchable strip. The stretchable strip is stretched to a size to be accommodated around, and to stick around, upper and lower lips of the user, surrounding the mouth but not touching the upper and lower lips of the user, such that the upper and lower lips are brought together by the tension created in the stretchable strip. Further, to obtain habitual mouth closure and nasal breathing, for many children and adults it is necessary to strengthen the orbicularis oris muscle. While wearing the nasal breathing training tape, the user feels the orbicular muscle oris contract, causing the lips to close. The tension created by the nasal breathing training tape, an overall neuro-sensory-motor system of the user is stimulated, so that the brain of the user learns to ‘switch on’ the nasal breathing, when the upper and lower lips are gently held together by the stretchable strip. In addition, the tape can be worn during the day as the user can speak and open their mouth while wearing the tape.

In another aspect of the present invention, a method for imparting training to a user to breathe from the nose and not from the mouth, during sleep time or wakefulness is provided. The method includes stretching a stretchable strip of a nasal breathing training tape to a size to be accommodated around upper and lower lips of the user, and to create tension therein. The stretchable strip includes a cutout portion disposed along a substantially central location of the stretchable strip. Further, the method includes sticking the stretched stretchable strip, via an adhesive layer applied to a second surface, opposite to the first surface of the stretchable strip, around the upper and lower lips, surrounding the mouth but not touching the upper and lower lips of the user, such that the upper and lower lips are pressed together by the tension created in the stretchable strip. Due to the tension created by the nasal breathing training tape, an overall neuro-sensory-motor system of the user is stimulated, so that the brain of the user learns to ‘switch on’ the nasal breathing, when the upper and lower lips are gently held together.

In yet another aspect of the present invention, a nasal breathing training tape without a cutout is provided. Such nasal breathing training tape is also adapted to impart training to a user to breathe from the nose and not from the mouth, during sleep time or wakefulness. The nasal breathing training tape includes a stretchable strip and an adhesive layer. The stretchable strip includes a first surface, and a second surface opposite to the first surface. The stretchable strip is stretchable to create tension therein. Further, the adhesive layer may be applied to the second surface of the stretchable strip. The stretchable strip is stretched to a size to be accommodated and stuck over and beyond upper and lower lips, surrounding the mouth and completely covering the upper and lower lips of the user, such that the upper and lower lips are held together by the tension created in the stretchable strip. Further, due to the tension created by the nasal breathing training tape, an overall neuro-sensory-motor system of the user is stimulated, so that the brain of the user learns to ‘switch on’ the nasal breathing, when the upper and lower lips are completely covered to be held together by the stretchable strip. This together with the other aspects of the present invention, along with the various features of novelty that characterize the present invention, is pointed out with particularity in the claims annexed hereto and forms a part of the present invention. For a better understanding of the present invention, its operating advantages, and the specified object attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 illustrates an environment wherein a nasal breathing training tape is shown to be incorporated surrounding the users' mouth to impart training to a user to breathe from the nose and not from the mouth, during sleep time or wakefulness time, in accordance with an exemplary embodiment of the present disclosure;

FIGS. 2A to 2C illustrate various views of a nasal breathing training tape, i.e. FIG. 2A illustrates top view of the nasal breathing training tape, FIG. 2B illustrates back view of the nasal breathing training tape with an adhesive layer, and FIG. 2C illustrates back view of the nasal breathing training tape with a layer covering the adhesive layer which is peeled off to allow the nasal breathing strip to be affixed by way of the adhesive, in accordance with an exemplary embodiment of the present disclosure;

FIG. 3 illustrates an example of dimensional measurement of a nasal breathing training tape, in accordance with an exemplary embodiment of the present disclosure;

FIG. 4 illustrates an example of shape and dimensional measurement of a nasal breathing training tape, in accordance with another exemplary embodiment of the present disclosure;

FIG. 5 illustrates a nasal breathing training tape having patterns, in accordance with another exemplary embodiment of the present disclosure;

FIG. 6 illustrates a flow chart of a method of using a nasal breathing training tape, in accordance with another exemplary embodiment of the present disclosure;

FIG. 7 illustrates an environment wherein a nasal breathing training tape without a cutout is shown to be incorporated along the users' mouth to impart training to a user to breathe from the nose, and not from the mouth, during sleep time or wakefulness time, in accordance with an exemplary embodiment of the present disclosure;

FIGS. 8A to 8C illustrate various views of a nasal breathing training tape without a cutout, i.e. FIG. 8A illustrates a top view of the nasal breathing training tape, FIG. 8B illustrates a back view of the nasal breathing training tape with an adhesive layer, and FIG. 8C illustrates back view of the nasal breathing training tape with a layer covering the adhesive layer which is peeled off to allow the nasal breathing strip to be affixed by way of the adhesive, in accordance with an exemplary embodiment of the present disclosure;

FIG. 9 illustrates an example of dimensional measurement of a nasal breathing training tape, in accordance with an exemplary embodiment of the present disclosure;

FIG. 10 illustrates an example of shape and dimensional measurement of a nasal breathing training tape, in accordance with another exemplary embodiment of the present disclosure; and

FIG. 11 illustrates a nasal breathing training tape having patterns, in accordance with another exemplary embodiment of the present disclosure; and

Like reference numerals refer to like parts throughout the description of several views of the drawings.

### DETAILED DESCRIPTION OF THE INVENTION

For a thorough understanding of the present invention, reference is to be made to the following detailed description, including the appended claims, in connection with the above-described drawings. Although the present invention is described in connection with exemplary embodiments, the present invention is not intended to be limited to the specific forms set forth herein. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The terms, “a” and “an” herein do not denote a limitation of quantity, but rather denote the presence of at least one of the referenced items.

The present invention provides a nasal breathing training tape. The nasal breathing training tape is adapted to impart training to a user to breathe from the nose, and not from the mouth, during sleep time or wakefulness time. The nasal breathing training tape includes a stretchable strip, an adhesive layer, and a cutout portion. The stretchable strip includes a first surface, and a second surface placed on top of the first surface. The stretchable strip is stretchable to create tension therein. Further, the adhesive layer may be applied to the second surface of the stretchable strip. Furthermore, the cutout portion is configured on the stretchable strip at a substantially central location of the stretchable strip. The stretchable strip is stretched to a size to be accommodated and stuck around a upper and lower lips, surrounding the mouth but not touching the upper and lower lips, of the user, such that the upper and lower lips are held together by the tension created in the stretchable strip. Further, due to the tension created by the nasal breathing training tape, an overall neuro-sensory-motor system of the user is stimulated, so that the brain of the user learns to ‘switch on’ the nasal breathing, when the upper and lower lips are gently held together by the stretchable strip.

The present invention also provides a method for imparting training to a user to breathe from the nose, and not from the mouth, during sleep time or wakefulness time. The method includes stretching a stretchable strip of a nasal breathing training tape up to a size to be accommodated around upper and lower lips of the user, and to create tension therein. The stretchable strip includes a cutout portion configured on the stretchable strip along a substantially central location of the stretchable strip. Further, the method includes sticking the stretched stretchable strip, via an adhesive layer applied to a second surface and placed on top of a first surface of the stretchable strip, around the upper and lower lips, surrounding the mouth but not touching the upper and lower lips of the user, such that the upper and lower lips are held together by the tension created in the stretchable strip. Due to the tension created by the nasal breathing training tape, an overall neuro-sensory-motor system of the user is stimulated, so that the brain of the user learns to ‘switch on’ the nasal breathing, when the upper and lower lips are gently held together.

In yet another aspect of the present invention, a nasal breathing training tape without a cutout is provided. Such nasal breathing training tape is also adapted to impart training to a user to breathe from the nose, and not from the mouth, during sleep time or wakefulness time. The nasal breathing training tape includes a stretchable strip and an adhesive layer. The stretchable strip includes a first surface, and a second surface opposite to the first surface. The stretchable strip is stretchable to create tension therein. Further, the adhesive layer may be applied to the second surface of the stretchable strip. The stretchable strip is stretched to a size to be accommodated and stuck over and beyond upper and lower lips, surrounding the mouth and completely covering the upper and lower lips of the user, such that the upper and lower lips are held together by the tension created in the stretchable strip. Further, due to the tension created by the nasal breathing training tape, an overall neuro-sensory-motor system of the user is stimulated, so that the brain of the user learns to ‘switch on’ the nasal breathing, when the upper and lower lips are completely covered to be held together by the stretchable strip.

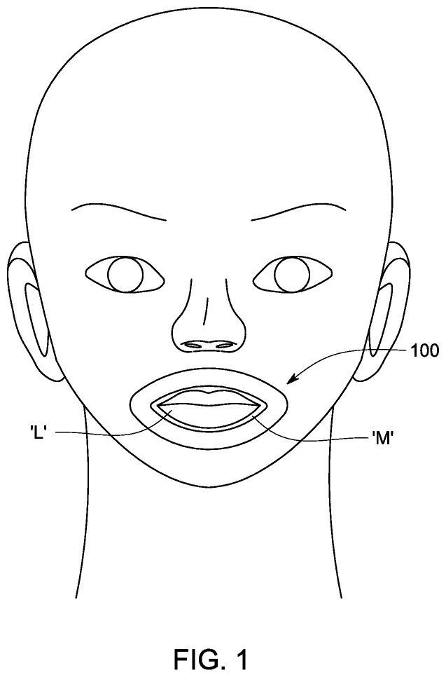

Referring now to FIG. 1, a nasal breathing training tape 100 is shown to be incorporated around a user's mouth ‘M’ to impart training to the user to breathe from the nose, and not from the mouth, during sleep time or wakefulness time, in accordance with an exemplary embodiment of the present disclosure. Specifically, nasal breathing training tape 100 may be stretched to a size to be accommodated and to stick around upper and lower lips surrounding the mouth ‘M’ but not touching the upper and lower lips of the user, such that the upper and lower lips 1′ are held together.

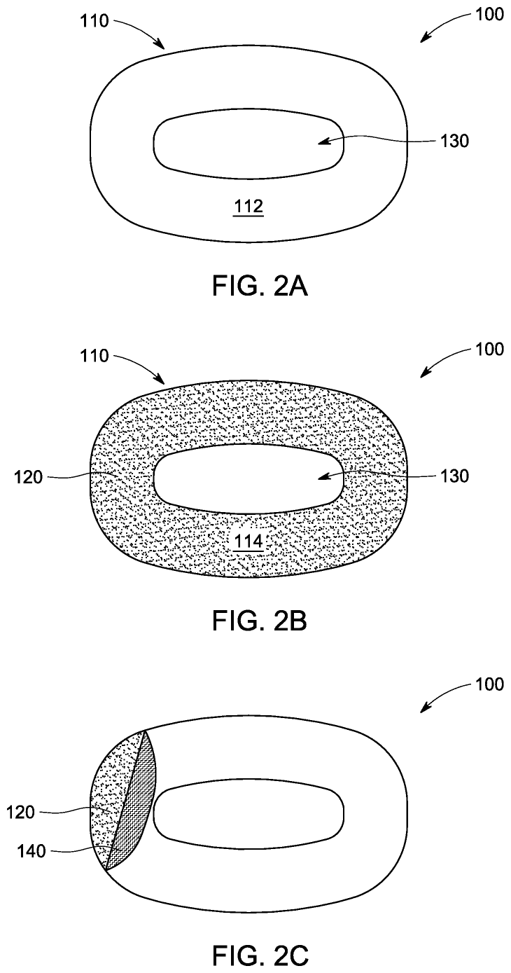

Referring now to FIGS. 2A to 2C, various views of the nasal breathing training tape 100, in accordance with an exemplary embodiment of the present disclosure are shown and now will be explained in conjunction with FIG. 1. In one example embodiment, as shown in FIGS. 2A to 2C, the nasal breathing training tape 100 may include a stretchable strip 110, an adhesive layer 120, and a cutout portion 130. The stretchable strip 110 may include a first surface 112, and a second surface 114 opposite to the first surface 112. The stretchable strip 110 may be stretchable to create tension therein. Further, the adhesive layer 120 may be applied to the second surface 114 of the stretchable strip 100. Furthermore, the cutout portion 130 may be disposed along a substantially central location of the stretchable strip 110. In one embodiment, the cutout portion 130 generally corresponds to the upper and lower lips ‘L’, such that the upper and lower lips 1′ protrudes from the cutout portion 130, as shown in FIG. 1.

The stretchable strip 110 may be stretched to a size to be accommodated and to be stuck around the upper and lower lips surrounding the mouth ‘M’ but not touching the upper and lower lips ‘L’, of the user, such that the upper and lower lips ‘L’ are held together by the tension created in the stretchable strip 110. Further, due to the tension created by the nasal breathing training tape 100, an overall neuro-sensory-motor system of the user is stimulated, so that the brain of the user learns to ‘switch on’ the nasal breathing, when the upper and lower lips 1′ are gently held together by the stretchable strip 110.

In one embodiment, the stretchable strip 110 may be made of a cotton material and/or a synthetic material that has a stretchable and elastic property. However, without departing from the scope of the present disclosure, the stretchable strip 110 may be made of any other suitable material having a stretchable and elastic property. In one preferred embodiment of the present disclosure, the stretchable strip 110 may include a property of stretchability of about 20% to about 30%. For example, the stretchable strip 110 may be stretched to about 20% to about 30% of its actual size to create tension in the stretchable strip 110. Stretchability may be determined as described below, based on a change in dimension when a force of 300 grams is applied from an original rest condition of the stretchable strip 110 until resistance is encountered to substantially stop the stretching. The adhesive layer 120 also retains its adhesiveness when the stretchable strip 110 is stretched to about 20% to 30%. When released, the stretchable strip 110 will recover most of its original dimensions under elastic property of the strip 110. In one example, a stretchable strip 110 of about 54 mm (in a direction from a left side to a right side of the lips, perpendicular to a mouth opening direction) may stretch to about 65 mm, at which the adhesiveness of the adhesive layer 120 is also effectively retained.

In one further embodiment, the stretchable strip 110 may be one of a rectangular shape or of an elliptical shape (with respect to an outer perimeter). One example depiction of the elliptical shape of the stretchable strip is shown in FIGS. 2A to 2C. However, the stretchable strip 110 may also be of the rectangular shape (with curved corners and straight sides), such as shown in FIG. 4. Where the stretchable strip 110 is rectangular, the corners thereof may be rounded, as shown in FIG. 4. Further, the cutout portion 130 may also correspond to the shape of the stretchable strip 110, and may be one of a rectangular shape, as shown in FIG. 4, and may be of an elliptical shape, as shown in FIGS. 2A-2C. Where the shape of the cutout portion 130 is rectangular, the corners thereof may also be rounded, as shown in FIG. 4. As shown in FIGS. 2A-2C or FIG. 4, the shape of the stretchable strip 110 and the cutouts 130 are elliptical or rectangular in these examples. However, the shape of the stretchable strip 110 and the cutouts 130 are not considered to be limited only to elliptical or rectangular. The disclosure, without departing from the scope of the present disclosure, intends to cover various other shapes, such as circular or square and so forth. However, the outer perimeter of the stretchable strip 110 and the shape of the cutout portion 130 will generally follow the shape of the upper and lower lips of the user.

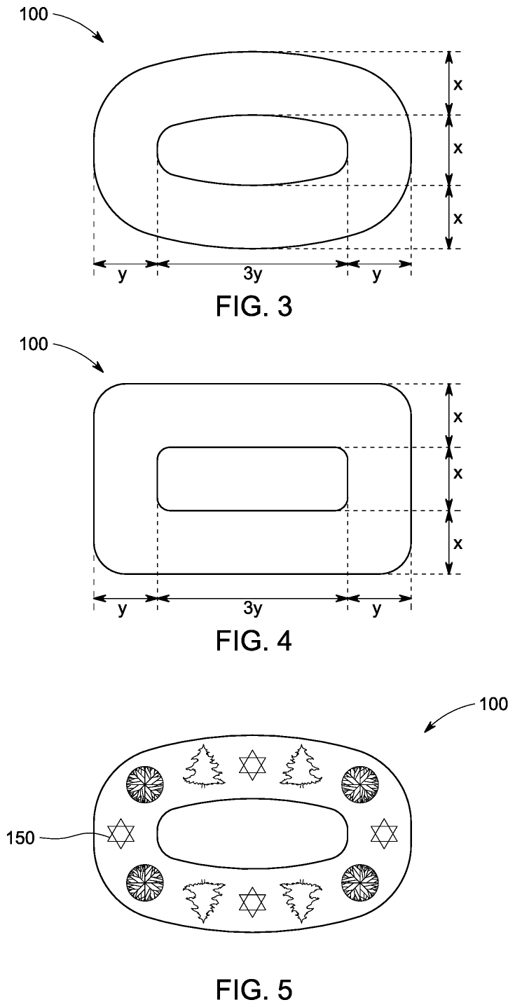

In one further embodiment, the stretchable strip 110 comprises a predetermined shape having a height (in a direction of opening and closing of the mouth) and a width (in a direction from left to right sides of the mouth of the user), wherein a ratio of height to width is about 3:5. For example, as shown in FIG. 3, the stretchable strip 110 has a predetermined shape of an ellipse having a height of 3x and a width of 5y, wherein x=y=1. Another example is shown in FIG. 4, where the stretchable strip 110 has a predetermined shape of a rectangle having a height of 3× and a width of 5y, wherein x=y=1. In one example, the stretchable strip 110 may have a width of about 54 mm and a height of about 33 mm. In one another example, the stretchable strip 110 may have a height of about 30 mm and the width of about 50 mm.

In one further embodiment, an area ratio of the cutout portion 130 to the stretchable strip 110 may be about 1:3, although other variations may be considered without departing from the scope of the present disclosure. For example, if the stretchable strip 110 has a width of 5y and height of 3x; then the cutout portion 130 may be of 1× in height and 3y in width, wherein x=y=1. In a further example, if the stretchable strip 110 has a width of about 54 mm and a height of about 33 mm, then the cutout portion 130 may have a width of about 32 mm and a height of about 11 mm. In a further example, if the stretchable strip 110 has a height of about 30 mm and a width of about 50 mm, then the cutout portion 130 may have a width of about 30 mm and a height of about 10 mm.

In embodiments, the cutout portion 130 has a width of between 30 mm and 45 mm and a height of between 10 mm and 20 mm. Different size devices may be provided depending on the size of the mouth, whether the wearer is adult or child, etc. The border of material around the cutout portion 130 should be about 20 mm to 35 mm when measuring from the cutout portion 130 to the outside of the stretchable strip 110 along a shortest perpendicular line (along linear portions) or radial line (at corner portions).

In embodiments, the stretchability described herein is determined by gently pulling the device until there is a clear resistance to further stretching and measuring the before and after dimensions. A more precise testing method is not required as precise stretchabilities are not being recited. The device should stretch between 110% and 140% at least in the width direction (in the direction of the left and right sides of the mouth in use) and optionally also in the height direction (the mouth opening and closing direction) when gently pulled by an adult user until resistance. The force applied may be 300 gram force.

The stretchable strip 110 is stretchable by the user in at least the horizontal direction and/or the vertical direction. The stretchable strip should additionally be elastic to apply a force/tension to the lips when used. In embodiments, the stretchable strip 110 is a fibrous material. In embodiments, the stretchable strip 110 is made of a blend of cotton and synthetic fiber. In one embodiment, cotton and spandex fibers are blended. Other blends include Cotton, Rayon and/or Nylon. The fibers may be woven. In one embodiment, the stretchability and elasticity are significantly greater in the horizontal direction than the vertical direction. This may be achieved by horizontally orienting a majority of the fibers. In one embodiment, cotton and synthetic threads are included into a yeft yarn to provide a uni-directional elasticity and stretch in a horizontal direction along the width of the mouth.

In one further embodiment, the stretchable strip 110 may include one or more patterns of cartoon characters, or, one or more universal characters including cars, stars, moon, sun, flowers, or any other graphical elements, and so forth, as shown in FIG. 5. Particularly, the first surface 112 of the stretchable strip 110 may include such cartoon characters and/or universal characters. Such cartoon characters and/or universal characters may make the nasal breathing training tape 100 attractive to children and children may get encouraged to stick the nasal breathing training tape for training purposes.

In one embodiment, as mentioned above, the adhesive layer 120 may be applied to the second surface 114 of the stretchable strip 110. The adhesive in the adhesive layer 120 is such that it is adaptable to human skin. In one embodiment, as shown in FIG. 2B, the nasal breathing training tape 100 may be directly stretched and stuck to surround the mouth ‘M’ but not touching the upper and lower lips with the help of an exposed adhesive layer 120. Such adhesive may retain its adhesiveness upon applying a liquid to the adhesive layer 120. However, in another embodiment, as shown in FIG. 2C, the adhesive layer 120 may be covered by another layer, such as layer 140, made of paper or plastic. In this embodiment, the user may remove the layer 140 to expose the adhesive layer 120. Upon exposure of the adhesive layer 120, the nasal breathing training tape 100 may be directly stretched and stuck to surround the mouth ‘M’ but not touching the upper and lower lips ‘L’.

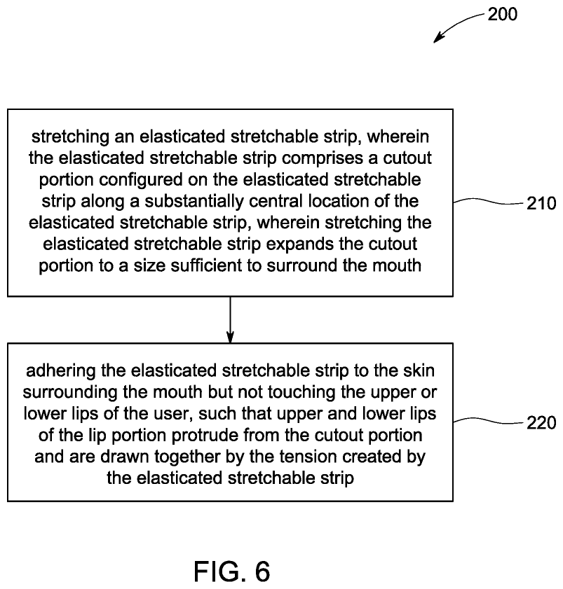

Referring now to FIG. 6, a method 200 for imparting training to the user to breathe from the nose, and not from the mouth, during sleep time or wakefulness time is provided. At step 210, the stretchable strip 110 of the nasal breathing training tape 100 is stretched to a size to be accommodated around upper and lower lips of the user, and to create tension therein. The tension is created by elastic properties of the tape. The tension may be created in the up and down direction (the opening and closing direction of the mouth), the left and right direction (across the mouth) or both. In one embodiment, the stretchable strip may be stretched from about 20% to about 30% additional to the original length. Further, at step 220, the method 200 includes sticking the stretched stretchable strip, via the adhesive layer 120, so as to surround the mouth but not touching the upper and lower lips, of the user, such that the upper and lower lips are held together by the tension created in the stretchable strip 110. Due to the tension created by the nasal breathing training tape 100, an overall neuro-sensory-motor system of the user is stimulated, so that the brain of the user learns to ‘switch on’ the nasal breathing, when the upper and lower lips are gently held together. In one embodiment, the nasal breathing training tape 100 may be worn during the day for about 30 minutes to two hours, especially while a child (or adult) is distracted. The nasal breathing training tape 100 may be worn during sleep for a more extended period of time.

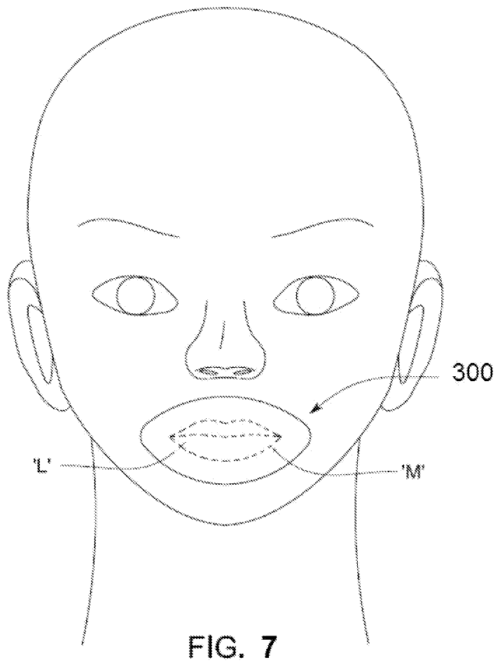

Referring now to FIGS. 7 to 11 to describe another embodiment of a nasal breathing training tape 300 without a cutout, such as the cutout 130 as shown in FIGS. 1 to 6. As shown in FIG. 7, an environment wherein the nasal breathing training tape 300 (without the cutout 130) is shown to be incorporated along a user's mouth ‘M’ to impart training to the user to breathe from the nose, and not from the mouth, during sleep time or wakefulness time, in accordance with an exemplary embodiment of the present disclosure. Specifically, nasal breathing training tape 300 may be stretched to a size to be accommodated and stuck over and beyond a upper and lower lips surrounding the mouth ‘M’ and completely covering the upper and lower lips of the user, such that the upper and lower lips 1′ held together.

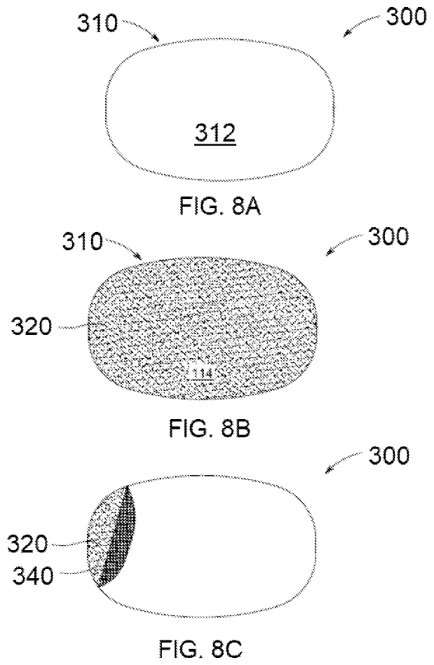

Referring now to FIGS. 8A to 8C, various views of the nasal breathing training tape 300, in accordance with an exemplary embodiment of the present disclosure are shown and now will be explained in conjunction to FIG. 7. In one example embodiment, as shown in FIGS. 8A to 8C, the nasal breathing training tape 300 may include a stretchable strip 310 and an adhesive layer 320. The stretchable strip 310 may include a first surface 312, and a second surface 314 opposite to the first surface 312. The stretchable strip 310 may be stretchable to create tension therein. Further, the adhesive layer 320 may be applied to the second surface 314 of the stretchable strip 300.

The stretchable strip 310 may be stretched to a size to be accommodated and stuck over and beyond the upper and lower lips surrounding the mouth ‘M’ and completely covering the upper and lower lips of the user, such that the upper and lower lips 1′ held together by the tension created in the stretchable strip 310. Further, due to the tension created by the nasal breathing training tape 300, an overall neuro-sensory-motor system of the user is stimulated, so that the brain of the user learns to ‘switch on’ the nasal breathing, when the upper and lower lips 1′ are completely covered to be held together by the stretchable strip 310.

In one embodiment, the stretchable strip 310 may be made of a cotton material or a synthetic material that has stretchable property therein. However, without departing from the scope of the present disclosure, the stretchable strip 310 may be made of any other suitable material having stretchable property. In one preferred embodiment of the present disclosure, the stretchable strip 310 may include a property of stretchability of about 20% to about 30%. For example, the stretchable strip 310 may be stretched to about 20% to about 30% of its actual size to create tension in the stretchable strip 310. The adhesive layer 320 also retains its adhesiveness when the stretchable strip 310 is stretched to about 20% to 30%. In one example, a perfect stretch for the stretchable strip 310 of about 54 mm may be about 65 mm, at which the adhesiveness of the adhesive layer 320 is also effectively retained.

In one further embodiment, the stretchable strip 310 may be of one of a rectangular shape or of an elliptical shape. One example depiction of the elliptical shape of the stretchable strip is shown in FIGS. 8A to 8C. However, the stretchable strip 310 may also be of the rectangular shape, such as shown in FIG. 10. The stretchable strip 310 where its shape is rectangular, the corners thereof may be rounded, as shown in FIG. 10. As shown in FIGS. 8A-8C or FIG. 10, the shape of the stretchable strip 310 shown to be elliptical or rectangular, are for better understanding of the present invention, and that the shape of the stretchable strip 310 may not considered to be limiting in any manner only to elliptical or rectangular. The disclosure, without departing from the scope of the present disclosure, intend to cover various other shapes, such as circular or square and so forth.

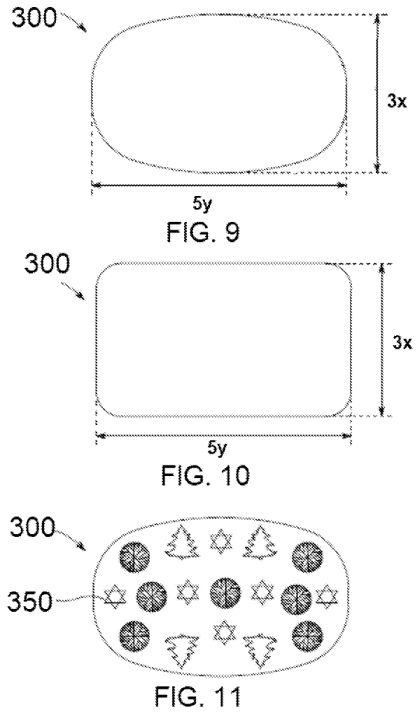

In one further embodiment, the stretchable strip 310 comprises a predetermined shape having a width and a length, wherein a ratio of width to length is about 3:5. For example, as shown in FIG. 9, the stretchable strip 310 has a predetermined shape of an ellipse having a width of 3x and a length of 5y, wherein x=y=1. Another example is shown in FIG. 10, where the stretchable strip 310 has a predetermined shape of a rectangle having a width of 3x and a length of 5y, wherein x=y=1. In One example, the stretchable strip 310 may have a length of about 54 mm and a width of about 33 mm. In one another example, the stretchable strip 310 may have a width of about 30 mm and the length of about 50 mm.

In one further embodiment, the stretchable strip 310 may include one or more patterns of cartoon characters, or, one or more universal characters including cars, stars, moon, sun, flowers, and so forth, as shown in FIG. 11. Particularly, the first surface 312 of the stretchable strip 310 may include such cartoon characters and/or universal characters. Such cartoon characters and/or universal characters may make the nasal breathing training tape 300 attractive to children and children may get encouraged to stick such nasal breathing training tape for training purpose.

In one embodiment, as mentioned above, the adhesive layer 320 may be applied to the second surface 314 of the stretchable strip 310. The adhesive in the adhesive layer 320 is such that it is adaptable to human skin. In one embodiment, as shown in FIG. 8B, the nasal breathing training tape 300 may be directly stretched and stuck over and beyond a upper and lower lips ‘L’, surrounding the mouth ‘M’ and completely covering the upper and lower lips 1′ of the user, with the help of an exposed adhesive layer 320. Such adhesive may retain its adhesiveness upon applying a liquid to the adhesive layer 320. However, in another embodiment, as shown in FIG. 8C, the adhesive layer 320 may be covered by another layer, such as layer 340, made of paper or plastic. In this embodiment, the user may remove the layer 340 to expose the adhesive layer 320. Upon exposure of the adhesive layer 320, the nasal breathing training tape 300 may be directly stretched and stuck over and beyond a upper and lower lips surrounding the mouth ‘M’ and completely covering the upper and lower lips 1′ of the user.

The present invention provides a nasal breathing training tape, such as the nasal breathing training tape 100 or 300, which offers the various advantages. Most importantly, the nasal breathing training tape 100, 300 imparts training skills to children, teen or adults to breathe from the nose, and not from the mouth, during sleep time or wakefulness time. The nasal breathing training tape is placed surrounding the lips, but not in direct contact with the lips. The nasal breathing training tape may be made from cotton or synthetic materials to stretch in two or four ways. Due to the tension created by the nasal breathing training tape, the overall neuro-sensory-motor system is stimulated, so that the brain learns to ‘switch on’ nasal breathing when the lips are gently held together. The nasal breathing training tape stimulates the skin, which is regulated by the trigeminal, which sandwiches the perioral muscles, including the orbicularis oris regulated by the facial muscles. The purpose of the nasal breathing training tape is to re-educate children and teenagers to nasal breathe. The nasal breathing training tape should be worn during the day for 30 minutes to two hours, especially while child is distracted. The tape may also be worn during sleep. The device described herein may additionally find application in sports to encourage wearers to breathe properly. Athletes can wear the tape while partaking in physical exercise. Since the tape surrounds the mouth, the athlete can drink from a bottle as well as communicate.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present invention.

What is claimed is:

1. A nasal breathing training tape to impart training to a user to breathe from the nose, and not from the mouth, during sleep time or wakefulness time, the nasal breathing training tape comprising:
a stretchable strip having a first surface, and a second surface opposite to the first surface, wherein the stretchable strip is stretchable to create tension therein;
an adhesive layer applied to the second surface; and
a cutout portion disposed in a substantially central location of the stretchable strip,
wherein the stretchable strip is stretchable to a size to be accommodated, and to be stuck, around upper and lower lips, surrounding the mouth, but not touching the upper and lower lips of the user, such that the upper and lower lips are held together by the tension created in the stretchable strip, and
whereby due to the tension created by the nasal breathing training tape, an overall neuro-sensory-motor system of the user is stimulated, so that the brain of the user learns to ‘switch on’ the nasal breathing, when the upper and lower lips are gently held together by the stretchable strip.

2. The nasal breathing training tape of claim 1, wherein the stretchable strip comprises at least one of a cotton material and a synthetic material, having a stretchable and elastic property.

3. The nasal breathing training tape of claim 1, wherein the stretchable strip comprises one of a rectangular shape and an elliptical shape, wherein the stretchable strip with the rectangular shape has rounded corners.

4. The nasal breathing training tape of claim 1, wherein the stretchable strip comprises a predetermined shape having a height and a width, wherein a ratio of height to width is about 3:5.

5. The nasal breathing training tape of claim 1, wherein the stretchable strip comprises a stretchability of about 20% to about 30% additional to an unstretched dimension.

6. The nasal breathing training tape of claim 1, wherein the first surface of the stretchable strip comprises one or more graphical elements such as patterns of cartoon characters, or, one or more universal characters including cars, stars, moon, sun, flowers, and so forth.

7. The nasal breathing training tape of claim 1, wherein the cutout portion corresponds to the upper and lower lips such that the upper and lower lips protrude from the cutout portion.

8. The nasal breathing training tape of claim 1, wherein an area ratio of the cutout portion to the stretchable strip is about 1:3.

9. The nasal breathing training tape of claim 1, wherein the cutout portion comprises one of a rectangular shape and an elliptical shape, wherein the cutout portion with the rectangular shape comprises rounded corners.

10. The nasal breathing training tape of claim 1, wherein the adhesive is adaptable to human skin in that it is not allergen causing.

11. A method for imparting training to a user to breathe from the nose, and not from the mouth, during sleep time or wakefulness time, the method comprising:
stretching a stretchable strip of a nasal breathing training tape to a size to be accommodated around upper and lower lips of the user, and to create tension on the lips, the stretchable strip having a cutout portion disposed in a substantially central location of the stretchable strip; and
sticking the stretched stretchable strip, via an adhesive layer applied to a second surface, opposite to a first surface of the stretchable strip, around the upper and lower lips, surrounding the mouth but not touching the upper and lower lips of the user, such that the upper and lower lips are biased together by the tension created in the stretchable strip,
whereby due to the tension created by the nasal breathing training tape, an overall neuro-sensory-motor system of the user is stimulated, so that the brain of the user learns to ‘switch on’ the nasal breathing, when the upper and lower lips are gently held together.

12. The method of claim 11, wherein the stretchable strip is stretched to about 20% to about 30% additional to an unstretched dimension.

13. The method of claim 11, wherein the stretched stretchable strip is stuck around the upper and lower lips for about 30 minutes to 2 hours.

14. The method of claim 11, wherein the stretchable strip comprises at least one of a cotton material and a synthetic material, having a stretchable and elastic property.

15. A nasal breathing training tape to impart training to a user to breathe from the nose, and not from the mouth, during sleep time or wakefulness time, the nasal breathing training tape comprising:
a stretchable strip having a first surface, and a second surface opposite to the first surface, wherein the stretchable strip is stretchable to create tension therein;
an adhesive layer applied to the second surface; and
wherein the stretchable strip is stretched to a size to be accommodated and stuck over and beyond a upper and lower lips, surrounding the mouth and completely covering the upper and lower lips of the user, such that the upper and lower lips are held together by the tension created in the stretchable strip, and
whereby due to the tension created by the nasal breathing training tape, an overall neuro-sensory-motor system of the user is stimulated, so that the brain of the user learns to ‘switch on’ the nasal breathing, when the upper and lower lips are completely covered to be held together by the stretchable strip.
